(19) United States
(12) Patent Application Publication
(10) Pub. No.: US 2006/0246096 A1
(43) Pub. Date: Nov. 2, 2006

(54) Nanoparticles from biopolymers

(75) Inventors: Levente Novak, Debrecen (HU); Eva Fleischer, Dedrecen (HU); Janos Borbely, Debrecen (HU)

Correspondence Address: Thomas A. O'Rourke;Bodner & O'Rourke, 425 Broadhollow Road, Melville, NY, 11747, US

(73) Assignee: University of Debrecen, Department of Colloid and Environmental Chemistry

(21) Appl. No.: 11/406,208

(22) Filed: Dec. 29, 2004

Related U.S. Application Data
(60) Provisional application No. 60/532,989, filed on Dec. 29, 2003.

(51) Int. Cl. A61K 38/43; A61K 9/00

(52) U.S. Cl. 424/400; 424/94.1

(57) ABSTRACT

Methods are disclosed for preparing core-shell polymers of poly-γ-glutamic acid. The final products of the present invention are useful as drug delivery systems, for gene therapy, magnetic resonance imaging as well as in encapsulation technology. A method of forming a poly-γ-glutamic acid (PGA) useful in the formation of core shell polymers is disclosed. According to the method a PGA is prepared by fermentation with a suitable microorganism, capable of producing PGA in a suitable fermentation medium, under conditions and time appropriate for the microorganism used. The resulting culture medium is treated with centrifugation, to separate the cells from the PGA. Acetone is used to treat the resulting cell-free liquid to obtain the PGA from the fermentation medium. The obtained PGA is purified by dialysis and freeze drying the PGA. A method of forming a core shell polymer of a poly-γ-glutamic acid (PGA) is also disclosed. The core is formed by partially amidating a poly-γ-glutamic acid (PGA) by reaction with a diamino compound to at least partially cross-link the PGA. The outer shell of the core shell polymer is formed from a hydrophilic or a hydrophobic polymer.

This is a conversion of provisional patent application Ser. No. 60/532,989 filed Dec. 29, 2003 the disclosures of which are incorporated by reference.

### REFERENCES CITED

U.S. Patent Documents

U.S. Pat. No. 5,298,559 * Fujii et al.
U.S. Pat. No. 5,324,780 * Oshima et al.
U.S. Pat. No. 5,451,641 * Eisenhart et al.
U.S. Pat. No. 5,453,458 * Takeuchi et al.
U.S. Pat. No. 6,165,563 * Chandran et al.
U.S. Pat. No. 6,326,511 * Borbely J.

### SUMMARY OF THE INVENTION

The present invention relates to core-shell polymers of poly-γ-glutamic acid (PGA). The core is prepared of cross-linked poly-γ-glutamic acid compounds, that are prepared by amidizing PGA with diamino compounds. The core and the shell could be hydrophilic, hydrophobic or amphiphilic.

### DETAILED DESCRIPTION

The starting material of the present invention is PGA which is prepared by fermentation with a suitable microorganism, capable of producing PGA in a suitable fermentation medium, under conditions and time appropriate for the microorganism used.

The resulting culture medium is treated, by centrifugation, to separate the cells from the PGA. The resulting cell-free liquid is treated with acetone to obtain the PGA from this fermentation medium.

After this, the obtained PGA was purified by dialysis and freeze dried.

The molecular weight of the PGA is about 1,000,000.

The PGA is then partially amidated by reaction with a diamino compound. The diamino compound could be:
NH₂—CH₂—CH₂—(O—CH₂—CH₂)n-NH₂ n=2 to 12

EDBEA (2,2′-(Ethylenedioxy)bis(ethylamine)

This reaction determines the cross-linking of PGA. This cross-linking was performed in different proportions, from 1 to 99%.

This reaction takes place in water, in the presence of a water soluble carbodiimide (CDI) (eg., 1-[3-(dimethylamino)propyl]-3-Ethylcarbodiimide hydrochloride, etc).

The core is formed by crosslinking the PGA. In the second stage, the outer shell is formed from hydrophilic or hydrophobic polymer that optionally may be cross-linked a priori. The core-shell morphology may be a result of self assemble of modified PGA in the case when hydrophobic side chains ate introduced. Therefore, in aqueous solution the hydrophobic 0 chains are in the inner part and the residual hydrophilic segments occupy the corona. These particles are design for solubilization of water insoluble compounds or drugs.

The core and the outer shell can be formed independently to be hydrophobic or hydrophilic. The reaction takes place in a multi-step process. The formation of the primary crosslinked core is followed by formation of a covalently attached shell. The reaction is controlled by the conditions of reaction (e.g., concentration) to obtain either a very slightly crosslinked core wherein the porosity is very high, or to obtain a very highly crosslinked core wherein the porosity is very low. The degree of cross-linking could be in the range of from about 1% to about 99%.

In the present invention the core shell polymers of poly-γ-glutamic acid can be formed from the following ingredients:

| | | |
|---|---|---|
| Glutamic acid | 10-100 | g |
| Citric acid | 20-120 | g |
| Glycerol | 200-300 | g |
| NH₄Cl | 5-35 | g |
| K₂HPO₄ | .25-2.5 | g |
| MgSO₄*7H₂O | .25-2.5 | g |
| CaCl₂*2H₂O | .25-2.5 | g |
| MnSO₄*H₂O | .05-.40 | g |
| FeSO₄*7H₂O | .05-.40 | g |

In a preferred embodiment the range can be

| | | |
|---|---|---|
| Glutamic acid | 30-80 | g |
| Citric acid | 40-100 | g |
| Glycerol | 220-280 | g |
| NH₄Cl | 10-30 | g |
| K₂HPO₄ | .75-2.0 | g |
| MgSO₄*7H₂O | .75-2.0 | g |
| CaCl₂*2H₂O | .35-1.5 | g |
| MnSO₄*H₂O | .10-.35 | g |
| FeSO₄*7H₂O | .07-.20 | g |

A more preferred embodiment has the following composition:

| | | |
|---|---|---|
| Glutamic acid | 40-70 | g |
| Citric acid | 70-90 | g |
| Glycerol | 230-250 | g |
| NH₄Cl | 18-23 | g |
| K₂HPO₄ | 1.0 g-1.75 | g |
| MgSO₄*7H₂O | 1.0 g-1.75 | g |
| CaCl₂*2H₂O | .4 g-.5 | g |
| MnSO₄*H₂O | .2 g-.3 | g |
| FeSO₄*7H₂O | .1 g-.18 | g |

The pH may be in the range of 6.8 to 7.8

The core polymer may be formed by taking a quantity of the PGA formed in the manner described above in water, adding 0.01 to about 0.7 CDI and stirring for a suitable period of time. To the resulting solution a diamine compound is added. The diamino may be present in a range of 5 to 400 μl. In-a preferred embodiment about 10-250 μl diamine may be added depending on the diamine selected.

### EXAMPLE 1

Preparation of Poly-γ-Glutamic Acid

A solution was prepared by dissolving the following ingredients in 3 liters of distilled water.

| | |
|---|---|
| L-glutamic acid | 60 g |
| Citric acid | 78.8 g |
| Glycerol | 240 g |
| NH₄Cl | 21 g |
| K₂HPO₄ | 1.5 g |
| MgSO₄*7H₂O | 1.5 g |
| CaCl₂*2H₂O | 0.45 g |
| MnSO₄*H₂O | 0.24 g |
| FeSO₄*7H₂O | 0.14 g |

The pH was adjusted to 7.4 with NaOH. The medium was autoclaved.

The Bacillus licheniformis suspension was used to inoculate the flasks which contain the medium solution, and they were incubated on the shaker (150 rpm) for seven days, at 37 C. The contents of the culture flasks were centrifuged to separate the cells from the polymer solution. Two volumes of 99.5% acetone were added slowly to the supernatant liquid while stirring. The liquid was decanted and the precipitated polymer was dissolved in distilled water. The resulting polymer solution was dialyzed 1 day against EDTA solution, and 6 days against distilled water and freeze dried.

### EXAMPLE 2

Preparation of hydrophilic core polymer (10% of the free carboxyl groups are reacting) To a 10 g/l of 0.2 g of the PGA from Example 1 in water, 0.0433. CDI was added, and stirred 30 minutes. To the resulting solution 11.32 μl EDBEA was added, and stirred at ambient temperature for 24 hours. After this time, the resulting polymer solution was dialysed 7 days against distilled water and freeze dried.

### EXAMPLE 3

Preparation of hydrophilic core polymer (50% of the free carboxyl groups are reacting) To a 10 g/l of 0.2 g of the PGA from Example 1 in water, 0.2164 CDI was added, and stirred 30 minutes. To the resulting solution 56.6 μl EDBEA was added, and stirred at ambient. temperature for 24 hours. After this time the resulting polymer solution was dialysed 7 days against distilled water and freeze dried.

### EXAMPLE 4

Preparation of hydrophobic core polymer (50% of the free carboxyl groups are reacting)

To a 10 g/l of 0.5 g of the PGA from Example 1 in water, 0.5391 g CDI was added, and stirred 30 minutes. To the resulting solution 191.2 μl butylamine was added and stirred at ambient temperature for 24 hours. After this time the resulting polymer solution was dialysed 7 days against distilled water and freeze dried.

### EXAMPLE 5

Preparation of hydrophobic core polymer (50% of the free carboxyl groups are reacting) To a 10 g/l of 0.5 g of the PGA from Example 1 in water, 0.5391 g CDI was added and stirred 30 minutes. To the resulting solution 211.5 μl benzylamine was added, and stirred at ambient temperature for 24 hours. After this time, the resulting polymer solution was dialysed 7 days against distilled water and freeze dried.

We claim:

1. A method of forming a poly-γ-glutamic acid (PGA) useful in the formation of core shell polymers comprising:
preparing a PGA by fermentation with a suitable microorganism, capable of producing PGA in a suitable fermentation medium, under conditions and time appropriate for the microorganism used;
treating the resulting culture medium by centrifugation, to separate the cells from the PGA;
treating the resulting cell-free liquid with acetone to obtain the PGA from the fermentation medium;
purifying the obtained PGA by dialysis and freeze drying the PGA.

2. A method of forming a core shell polymer of a poly-γ-glutamic acid (PGA) comprising
forming the core by partially amidating a poly-γ-glutamic acid (PGA) by reaction with a diamino compound to at least partially cross-link the PGA;
forming the outer shell of the core shell polymer from a hydrophilic polymer

3. A method of forming a core shell polymer of a poly-γ-glutamic acid (PGA) comprising
forming the core by partially amidating a poly-γ-glutamic acid (PGA) by reaction with a diamino compound to at least partially cross-link the PGA;
forming the outer shell of the core shell polymer from a hydrophobic polymer.

4. The method according to claim 2 wherein the hydrophilic polymer is cross-linked.

5. The method according to claim 3 wherein the hydrophilic polymer is cross-linked.

6. The method according to claim 2 wherein the PGA is amidated with NH₂—CH₂—CH₂—(O—CH₂—CH₂)n-NH₂ and wherein n=2 to 12.

7. The method according to claim 3 wherein the PGA is amidated with NH₂—CH₂—CH₂—(O—CH₂—CH₂)n-NH₂ and wherein n=2 to 12.

8. The method according to claim 2 wherein the PGA is amidated with EDBEA (2,2′-(Ethylenedioxy)bis(ethylamine).

9. The method according to claim 3 wherein the PGA is amidated with EDBEA (2,2′-(Ethylenedioxy)bis(ethylamine).
